(19) United States
(12) Patent Application Publication
(10) Pub. No.: US 2025/0302717 A1
(43) Pub. Date: Oct. 2, 2025

(54) Systems and Methods for Making Personal Care Products

(71) Applicant: Bioeutectics Corporation, Wilmington, DE (US)

(72) Inventors: Tomás Silicaro, Nordelta (AR); Sergio David Pasini Cabello, Ciudad Mendoza (AR); Maria Romina Canales, Junín mendoza (AR)

(73) Assignee: Bioeutectics Corporation, Wilmington, DE (US)

(21) Appl. No.: 19/095,795

(22) Filed: Mar. 31, 2025

Related U.S. Application Data
(60) Provisional application No. 63/572,311, filed on Mar. 31, 2024.

(51) Int. Cl. A61K 8/44; A61K 8/34; A61K 8/365; A61K 8/60; A61K 8/9767; A61K 8/9789; A61Q 13/00; A61Q 17/00; A61Q 19/00

(52) U.S. Cl. CPC A61K 8/44; A61K 8/345; A61K 8/365; A61K 8/602; A61K 8/9767; A61K 8/9789; A61Q 13/00; A61Q 17/005; A61Q 19/00; A61K 2800/10

(57) ABSTRACT

Maintaining or enhancing the solubility and stability of natural products in personal products is a desirable characteristic that can be attained by using natural eutectic solvents (NADES). The solubility and stability is shown at a range of concentration levels. Being able to enhance the solubility and/or stability of these natural products such as essential oils and/or fragrances will lead to improved personal care products. These newly formulated personal care products will not only possess the requisite solubility and stability of available products but the use of NADES compositions will also lower the toxicity of the products, thereby making them attractive targets for use and for further study.

The present application claims priority under 35 USC 119 (e) to U.S. Provisional Application No. 63/572,311 filed Mar. 31, 2024, the contents of which are hereby incorporated by reference in its entirety.

### FIELD OF THE INVENTION

The present invention relates to enhancing the solubility of essential oils within natural deep eutectic solvents (NADES) across various concentration levels. The present invention also relates to solubilizing the mixture of essential oils in eutectic systems (oil/ES) in water at different level concentrations. Moreover, the present invention relates to assessing the stability of the studied mixtures.

### BACKGROUND OF THE INVENTION

Deep eutectic solvents or DESs are solutions of Lewis or Brønsted acids and bases that form a eutectic mixture. Deep eutectic solvents are highly tunable by varying the structure of the components or by varying the relative ratios of various components in the mixture. Because these are complicated systems that have widely varying properties, they have a wide variety of potential applications, including their use in catalysis, separation techniques, and electrochemical processes. The parent components of deep eutectic solvents tend to engage in complex hydrogen bonding networks, which means that the mixture tends to have significant freezing point depressions relative to the parent compounds/components in the mixture. Sometimes the individual components in the mixture may be solids at room temperature and atmospheric pressure, but when they are mixed together at room temperature and atmospheric pressure, the mixture may be a liquid that has a severely depressed freezing point (e.g., 10° C.).

The term “eutectic” was first coined in 1884 by British chemist and physicist Frederick Guthrie. The first generation of eutectic solvents were based on mixtures of quaternary ammonium salts with hydrogen bond donors such as amines and/or carboxylic acids. Natural deep eutectic solvents (NADES) are biologically based deep eutectic solvents which are composed of two or more compounds that are generally plant based primary metabolites, i.e., organic acids, sugars, alcohols, amines and amino acids. Water may also be present as part of the solvent, as water is sometimes difficult to remove due to its inability to be easily evaporated.

Much of the study of eutectic solvents since Frederick Guthrie coined the term “eutectic” has involved solvent mixtures wherein at least one of the components is a metal-based solvent. However, the discharge of metals from these solvent systems has demonstrated many of the drawbacks associated with metal leaching, and its associated health, environmental, and safety related issues. Accordingly, there has been some recent interest in non-metal containing eutectic systems.

The plurality of uses for which eutectic solvents can be used is constantly developing. The below demonstrates more potential uses for these eutectic solvent systems.

### BRIEF SUMMARY OF THE INVENTION

The present invention relates to enhancing the solubility of essential oils within natural deep eutectic solvents (NADES) across various concentration levels. Being able to enhance the solubility of these essential oils will lead to products that can be used in personal care products. The present invention also relates to solubilizing the mixture of essential oil/ES in water at different concentration levels. Moreover, the present invention relates to assessing the stability of the studied mixtures. Stability is one of several factors that one tries to attain to create personal care products.

### BRIEF DESCRIPTION OF THE SEVERAL VIEWS OF THE DRAWING

FIG. 1 depicts the results of sample test tubes showing BcC (left) and CGIH (right)/Fragrance A mixture dissolved at a concentration of 30% in water.

FIG. 2 depicts the results of a sample test tube showing BcC/Fragrance A mixture dissolved at 50% in water.

FIG. 3 depicts the results of sample test tubes showing BcC/Fragrance A (20%) mixture dissolved at 50% in water compared with samples that contain dipropylene glycol (DPG) and Ethanol.

FIG. 4 depicts the results of a sample test tube showing BcC/Fragrance B mixture at 50% in water

FIG. 5 depicts the results of sample test tubes showing NADES/Fragrance B mixture at 20% in water can compared with the clear mixture that was combined with water at a 10% concentration.

FIG. 6 depicts the results of a sample test tube showing BcC/Fragrance C mixture at 10% in water.

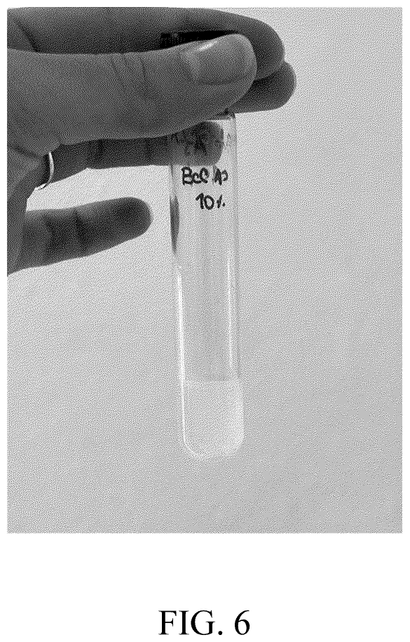

FIG. 7 depicts body splash formulation (left) with Dipropylenglicol (right) with BcC.

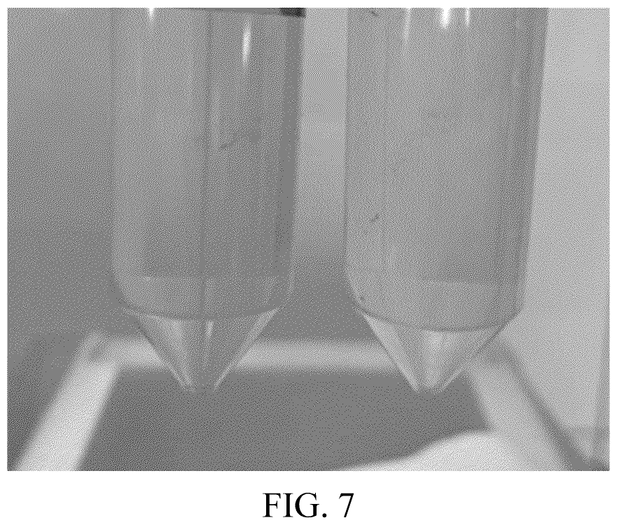

FIG. 8 shows the solubilization of Essential Oil A at 10% v/v using EtCiL, BcC, CDg, DgGl, and CDgGl. These results are compared to dipropylene glycol (DPG).

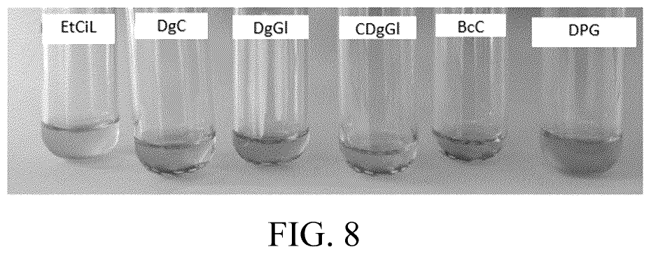

FIG. 9 shows the solubilization of Essential Oil B at 10% v/v, using EtCiL, BcC, CDg, DgGl, and CDgGl. These results are compared to dipropylene glycol (DPG).

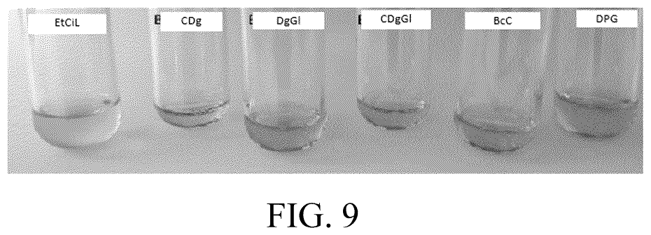

FIG. 10 shows the solubilization of Essential Oil C at 10% v/v, using EtCiL, BcC, CDg, DgGl, and CDgGl. These results are compared to dipropylene glycol (DPG).

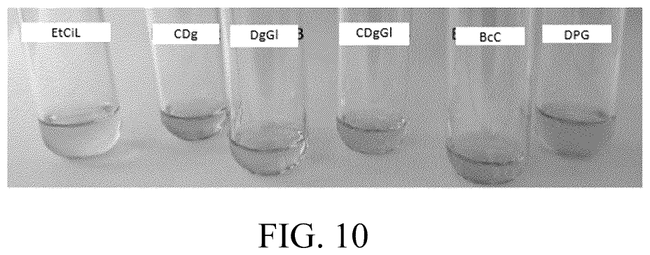

FIG. 11 shows the results of combining BcC, CDg, DgGl, and CDgGl with water to reach a final concentration of 1% v/v Essential Oil A. These results are then compared to dipropylene glycol (DPG).

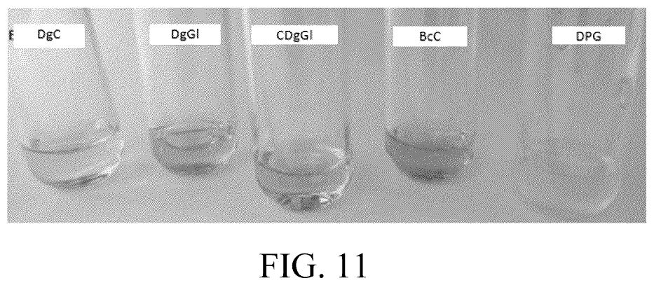

FIG. 12 shows the results of combining BcC, CDg, DgGI, and CDgGl with water to reach a final concentration of 1% v/v Essential Oil B. These results are then compared to dipropylene glycol (DPG).

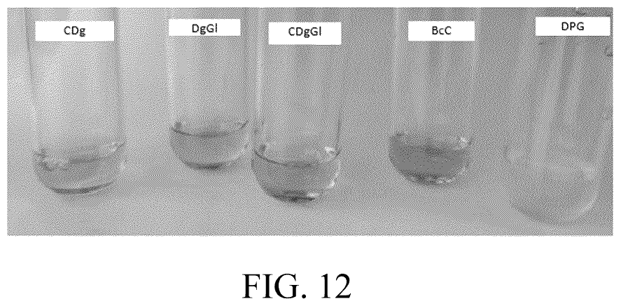

FIG. 13 shows the results of combining BcC, CDg, DgGl, and CDgGl with water to reach a final concentration of 1% v/v Essential Oil C. These results are then compared to dipropylene glycol (DPG).

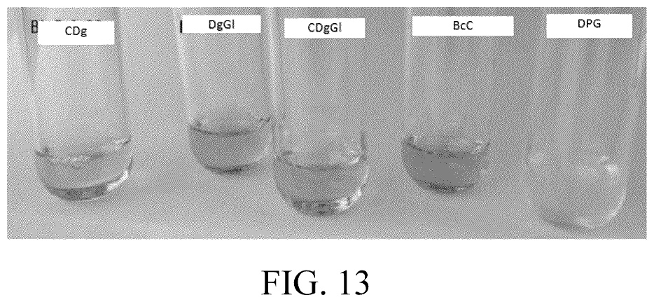

FIG. 14 depicts an infrared spectrum of BcC (coconut betaine, citric acid (molar ratio of 1:2)).

FIG. 15 depicts gasifier gels with basic NADES.

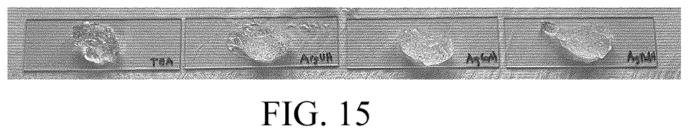

FIG. 16 depicts an image of the eau de parfum formulation with BcC A1 and the eau de parfum formulation with DPG A2.

FIG. 17 depicts an image of the body splash formulation with BcC A3 and the eau de parfum formulation with DPG A4.

FIG. 18 depicts the differences in visual perception and testing in a 3 dimensional access diagram for Eau de Parfum with DPG and with BcC.

FIG. 19 depicts the differences in visual perception and testing in a 3 dimensional access for Body Splash with DPG (blue) and BcC (red).

FIG. 20 depicts the differences in olfactory perception and testing in a 3 dimensional access diagram for Eau de Parfum with DPG and with BcC.

FIG. 21 depicts the differences in olfactory perception and testing in a 3 dimensional access for Body Splash with DPG (blue) and BcC (red).

FIG. 22 depicts a bar graph with the relative amounts for the various compounds found in eau de parfum formulation with BcC (A1) and the eau de parfum formulation with DPG (A2).

FIG. 23 depicts a bar graph with the relative amounts for the various compounds found in eau de parfum formulation with BcC (A1) and the eau de parfum formulation with 10% Ethanol (F4).

### DETAILED DESCRIPTION OF THE INVENTION

In an embodiment, the present invention relates to enhancing the solubility of essential oils within natural deep eutectic solvents (NADES) across various concentration levels as well as solubilizing essential oil/ES mixtures in water at different concentration levels. The goal was to use NADES and the enhanced solubilities to formulate personal care products with good stability.

Personal care products may comprise any of a number of products including products that may topically applied or ingested including but not limited to colognes, perfumes, mouthwashes, deodorants, toothpastes, hair gels, shampoos, conditioners, body washes, hair removal creams, lotions, other creams (such as hand creams, eye creams, and shaving creams), cleansers, eye drops, soaps, sunscreens, exfoliators, serum, and moisturizers. These topically applied products all have at least one active ingredient (some have several active ingredients) that provide the personal care product with some desired property. Desired properties include but are not limited to a desired fragrance, a desired viscosity, desired moisturization, desired cleansing, desired emulsification, desired stability, desired solubility, desired non-toxicity, desired smoothing ability, desired softening ability, desired anti-aging ability, desired wrinkle prevention ability, desired lubrication ability, desired anti-oxidant ability, desired lather ability, desired property allowing the active ingredient to be removed or retained on the applied site, desired protectant ability like UV blocking agents, desired graininess, desired anti-microbial ability (e.g., antibacterial, antifungal, antiviral), desired tint or color, desired opaqueness or transparency, desired thickening or lengthening ability, desired luster ability, desired antiperspirant or deodorant ability, desired sweat gland blocking ability, desired disease treating ability, desired grittiness or abrasiveness, desired flavor, desired evaporation or degradation characteristics, desired misting or nebulization ability, or other desired properties.

In a variation, the personal care products may also include products that are to be used alone or in conjunction with the above personal care products such as tooth brushes, hair brushes, other brushes or applicators, baby wipes, sanitary towels, make-up towels, or other towels, tissues, dental flosses, and razors. Many topical and orally administered products (including those that may be ingested) often contain toxic components. One aim of the present invention is to replace these toxic agents/solvents/products with NADES formulations that are less toxic but still deliver many of the desirable properties that the typically used (and toxic) solvent systems deliver.

NADES products were evaluated for their ability to be used as replacements for the traditionally used, toxic agents/solvents/products, including testing many of the desirable properties. For example, the immediately following experiments were performed in order to evaluate their ability to solubilize products and also evaluated for the ability to provide a stable formulation.

### Laboratory Test

The NADES products were prepared by stirring the various components in a container (such as a flask) using a magnetic stirrer at 40° C. for 30 min, with continuous stirring until the formation of a transparent solution was observed. The following ESs (eutectic solvent systems) were employed for the solubility tests:
DgL: decyl glucoside, lactic acid (molar ratio 1:0.36)
CDg: citric acid, dodecyl glucoside (molar ratio 1:1.4)
DgGl: decyl glucoside, glycerol (molar ratio 1:3.5)
DgGIC: decyl glucoside, glycerol, citric acid (molar ratio 1:1.8:2.1)
BcC: coconut betaine, citric acid (molar ratio of 4:1)

### Solubilization Procedure

For the solubility test, various diverse commercially available essential oils were blended with NADES to achieve a final volume of 2.5 mL in a glass tube, representing concentrations of 10%, 20%, and 30% of the essential oils. The mixtures were thoroughly vortexed and then subjected to an ultrasonic bath at room temperature for 15 minutes. The commercial essential oils tested were:
Fragrance A: Midnight Lavender
Fragrance B: Free Spirit
Fragrance C: Arabian Jasmine
Fragrance D: Fir bark
Fragrance E: Jack Pine
Fragrance F: Black Spruce
Fragrance G: Rose
   After 24 h, those mixtures that remained transparent and with only one phase were used for the mixture of NADES/essential oil with distilled water in increasing concentrations until phase separation was attained.
   The results of the mixtures with the oily fragrances A, B, and C are detailed in Table 1.

TABLE 1
Mixtures of NADES/essential oil at 10%

| NADES | Fragrance A | Fragrance B | Fragrance C |
|---|---|---|---|
| LGH | Emulsion | Emulsion | Emulsion |
| CGIH | Emulsion. | Emulsion with small | Emulsion. Opaque |
| | Clear mixture | bubbles. Clear mixture | mixture after US. |
| | after 24 h. | after 24 h. | |
| BeL | Emulsion. | Emulsion | Emulsion |
| CGlH | Emulsion. | Emulsion | Emulsion |
| ArgLH | Emulsion. | Emulsion | Emulsion |
| ArgCH | Emulsion. | Emulsion | Emulsion |
| BeUH | Emulsion. | Emulsion | Emulsion |
| BcC | Emulsion, | Emulsion, a | Emulsion, |
| | a lot of foam. | lot of foam. | some foam. |
| | Clear mixture | Clear mixture | Clear mixture |
| | after 24 h. | after 24 h. | after 24 h. |

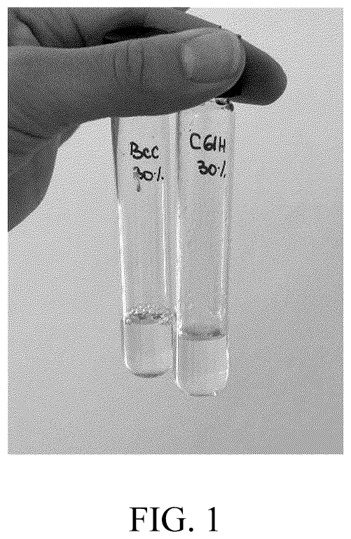

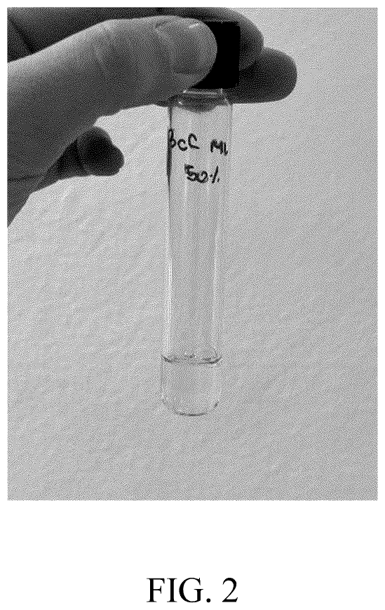

Based on the observations that are noted in Table 1, CGIH and BcC were selected for further testing with distilled water (Table 1). The maximum concentration achieved for the ES/essential oil mixture for Fragrance A was 30% for CGIH and 50% for BcC, equating to 3% and 5% of the essential oil, respectively (see FIG. 1 and FIG. 2 for photographs showing the results).

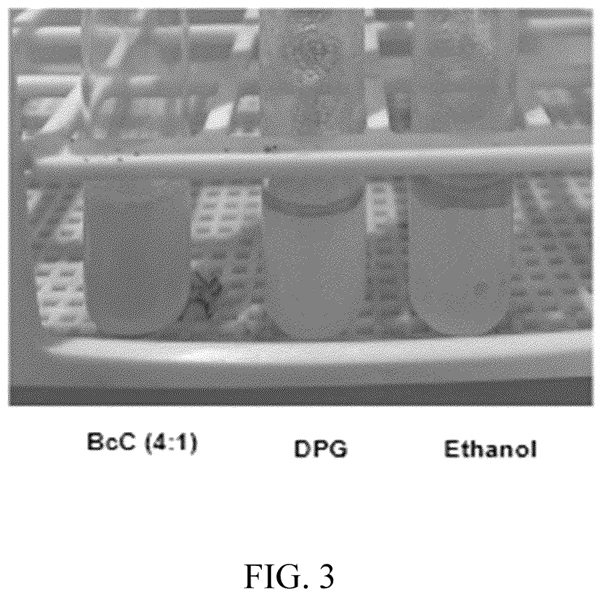

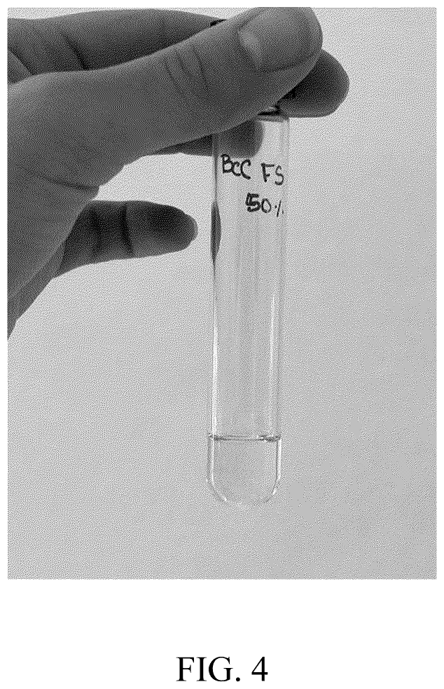

Regarding Fragrance B, the highest concentration attained for the NADES/fragrance B mixture was 20% for CGIH and 50% for BcC. Notably, only BcC produced a clear mixture after 24 hours (see FIG. 3 and FIG. 4 for photographs showing these results).

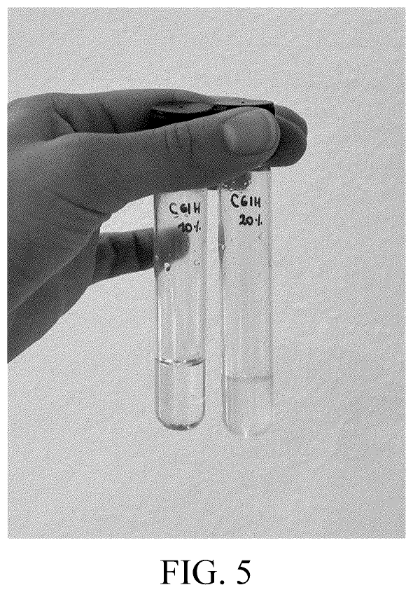

Referring to Fragrance C, only BcC formed a clear mixture after 24 h (Table 1). However, when this clear mixture was combined with water at a 10% concentration, it turned opaque and cloudy (see the photograph that is FIG. 5).

Several conclusions can be deduced from these experimental results.
CGIH and BcC solubilized the Fragrances A, B, and C at different level concentrations (between 20-50%) respectively.
   In the CGIH/Fragrance A mixture, it was possible to dissolve 30% of Fragrance A in water, whereas in the BcC/Fragrance A mix, Fragrance A could be dissolved at 50%.

This data demonstrates that there are at least several systems that are compatible with aqueous formulations, and these NADES are potential targets to be used as ingredients or raw materials in perfuming.

TABLE 1A
Mixtures of NADES with oils.

| | 10% v/v | | | | | | | 1% in water | | | | | | |
|---|---|---|---|---|---|---|---|---|---|---|---|---|---|---|
| Products | A | B | C | D | E | F | G | A | B | C | D | E | F | G |
| DgL | Yes | Yes | Yes | Yes | Yes | Yes | — | No | No | No | No | No | No | — |
| CDg | Yes | Yes | Yes | Yes | Yes | Yes | No | Yes | Yes | Yes | Yes | Yes | Yes | — |
| DgGl | Yes | Yes | Yes | Yes | Yes | Yes | Yes | Yes | Yes | Yes | Yes | Yes | Yes | No |
| DgGlC | Yes | Yes | Yes | Yes | Yes | Yes | Yes | Yes | Yes | Yes | Yes | Yes | Yes | Yes |
| BcC | Yes | Yes | Yes | Yes | No | No | — | Yes | Yes | Yes | Yes | — | — | — |
| DPG | Yes | Yes | Yes | Yes | Yes | Yes | Yes | No | No | No | No | No | No | No |

The results of the mixtures with the oily fragrances are detailed in Table 1.

Table 1A presents the solubility profiles of various Bioeutectics products under two distinct conditions: a 10% v/v concentration of oil in NADES and a 1% concentration of oil solubilized in NADES and then in water. The columns labeled A through G represent specific solubility tests or observations, with “Yes” indicating a positive result (e.g., complete dissolution) and “No” indicating a negative result (e.g., insolubility or phase separation). The table compares the performance of DgL, CDg, DgGI, DgGIC and BcC against DPG (dipropylene glycol). The data reveals variations in solubility across different NADES and concentrations, highlighting the influence of both the NADES composition and dilution on their behavior in aqueous solutions. Dashes (-) indicate that the test was not performed.

As can be seen, CDg, DgGl, and DgGIC demonstrate consistently positive results across all tests (A through G) at the 10% v/v concentration. This indicates robust solubility under these conditions. At 1% concentration, DgGl and DgGIC continue to show positive results across all tests. In contrast, DgL exhibits negative results in all tests (A through F) at the 1% concentration, suggesting poor solubility at this dilution. BcC shows a mix of positive and negative results at 10% v/v, and incomplete testing at 1% v/v, indicating variable or incomplete solubility.

### Incorporation of NADES as Additives in Perfuming

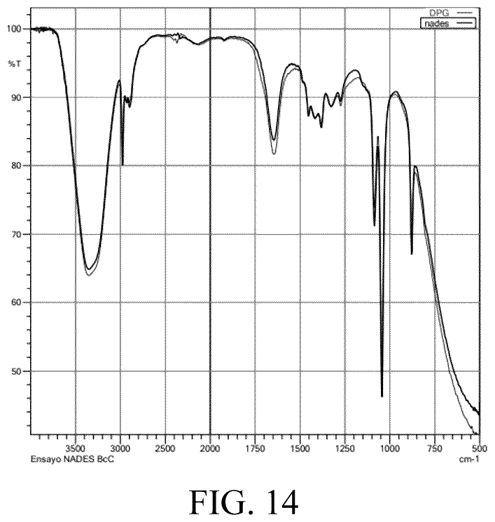

An experiment was performed to evaluate BcC as a perfuming additive. This NADES was incorporated into basic perfuming as body splash (about 4% oil essential), and Eau de parfum (15-25% oil essential) (see FIG. 14).

BcC was incorporated at concentrations commonly employed for DPG (dipropylene glycol), to explore its substitution in the formulation, at concentrations from 2-4% according to the formulation (see Table 2).

It was found that all perfuming formulations were stable and the aroma profile for the body splash and eau de parfum were maintained. DPG was successfully replaced by NADES using the BcC mixture.

TABLE 2
Basic fragrances formulation

| | Eau de parfum | | Body splash | |
|---|---|---|---|---|
| Components | A1 | A2 | A3 | A4 |
| Fragrance A | 15 | 15 | 3 | 3 |
| Midnight Lavender | | | | |
| BcC/DPG | 3 | 3 | 4 | 4 |
| Alcohol | 75 | 75 | 73 | 73 |
| Water | to 100 | to 100 | to 100 | to 100 |

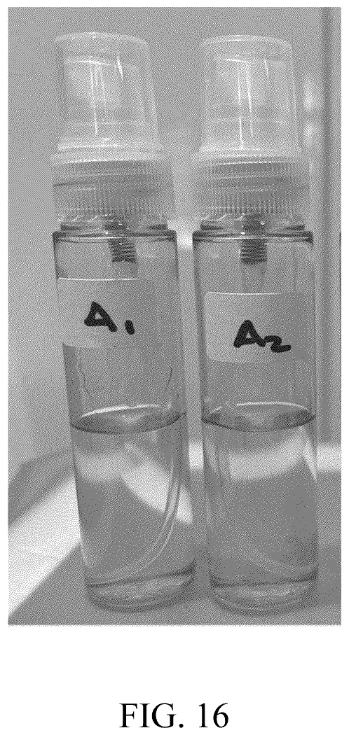

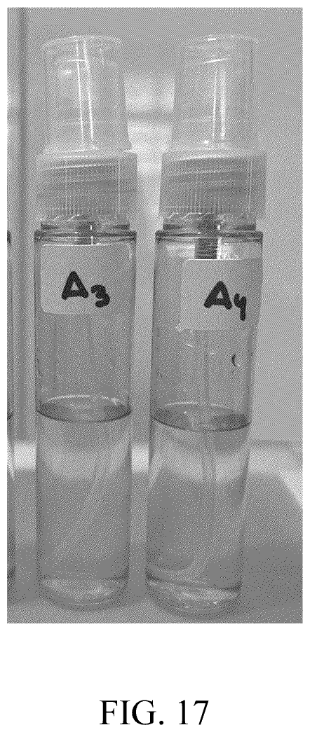

In conclusion, both fragrances as formulated were stable and the aroma profile was perceived and shown to be similar to the essential oil (see FIGS. 16 and 17). The DPG was successfully replaced by BcC in the same ratio (1:1 v/v). A1 shows the eau de parfum formulation with BcC and A2 shows the eau de parfum formulation with DPG. Similarly, A3 shows the body splash formulation with BcC and A4 shows the body splash formulation with DPG. In both instances the replacement of DPG with BcC gave similar profiles.

### Fragrances and Sensory Evaluation: “Perfume Testing”

The sensory evaluation of fragrances is a key parameter upon which one builds or confirms the good performance of a product, especially when the product is dedicated to beauty. The fragrance sector covers a wide range of products: fine fragrance, personal care, home care, and oral care and sensory evaluation is a key component that consumers look to when determining whether to buy and use that product. In an embodiment, odor quantification and/or characterization are needed to validate a change in a formula (to determine if the change is perceptible or not), to characterize the fragrance, and/or to establish links between the sensory qualities and the consumer data (e.g., whether the consumer will buy and use the product).

Two approaches (or a combination of the two approaches) can be followed to support and validate whether the sensory qualities will lead to sales and use:

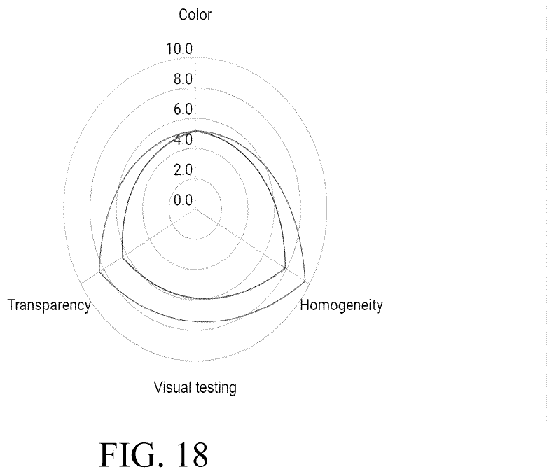

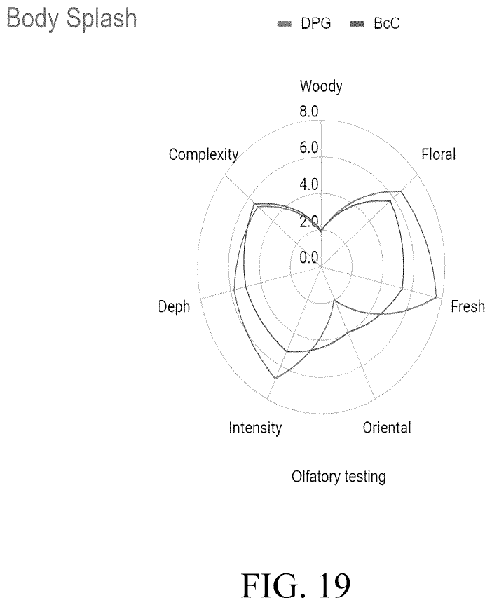

First, visual testing can be performed: In an embodiment, this assay comprises recording observations from inexperienced testers regarding color, homogeneity, and transparency and how it compares to the product prior to change. For example, note Eau de parfum (A1 and A2, FIG. 18) and Body Splash (A3 and A4, FIG. 19), which show the differences in visual perception and testing in a 3 dimensional access for Eau de Parfum (FIG. 18) with DPG (blue) and BcC (red) and for Body Splash (FIG. 19) with DPG (blue) and BcC (red). In both instances, the shape is similar and the colors are almost identical with the transparency and homogeneity only differing to a minor extent.

In both fragrances, which were compared with BcC and DPG as oil solubilization additives, the color change was not significant according to an analysis of variance (ANOVA). The homogeneity and transparency were barely significant in Body Splash, whose formulation has a higher water content and a lower essential oil content than the Eau parfume.

### Olfactory Testing was Also Performed.

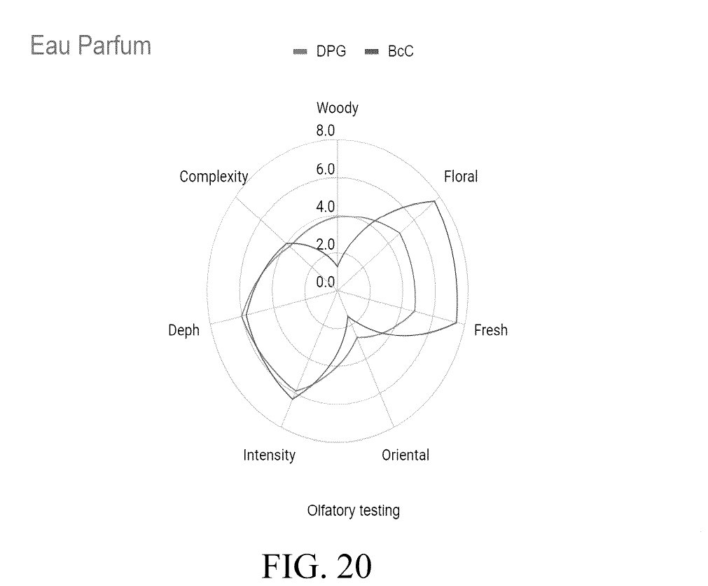

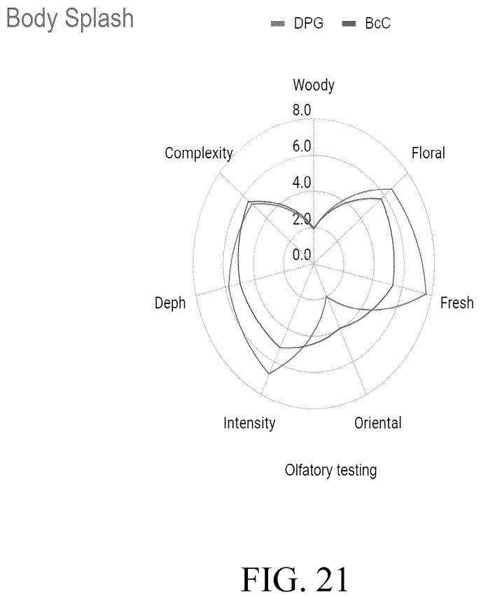

This assay consisted of recording and recognizing smell sensations from inexperienced testers, using the scent families from The Fragrance Wheel (established by Michael Edwards, an expert perfumer, and taxonomist): The various olfactory aromas that were tested include floral, fresh, oriental and woody aromas. Moreover, the identification of other characteristics such as complexity, depth and intensity were also tested. The results of the tests are shown in FIGS. 20 (Eau de Parfume) and 21 (Body Splash) with the DPG containing formulation shown in blue and the BcC containing formulation shown in red in both FIGS. 20 and 21.

The olfactory testing diagram for Eau de Parfum formulation demonstrated that the floral and fresh fragrances are highlighted with the BcC additive.

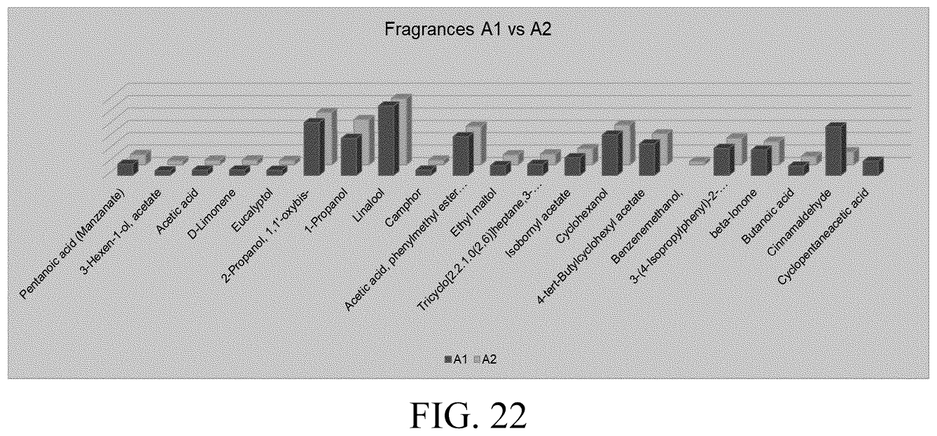

In both formulations, no significant differences were observed concerning complexity, depth, and intensity. The inexperienced testers perceived the same aroma profile, in concordance with the fragrance A. The relative amounts of natural products as found in gas chromatography analysis present in the Eau de Parfume with BcC (green) and DPG (yellow) are shown in FIG. 22, and the differences for the most part are insignificant indicating that BcC may be a good substitute for DPG.

### Replacing Ethanol in Different Perfume Formulations

### Objectives

In an embodiment, tests were performed to ascertain the results that could be attained when a substitution of ethanol with natural eutectic solvents (NADES) was made at different concentration levels in the formulation of perfumes with different fragrances.
In an embodiment, one objective was to evaluate the stability of the mixtures studied.

### Products Preparation

The selected NADES were those that tested positive in a solubility analysis of essential oils in different fragrances.

NADES products were prepared by stirring on a magnetic stirrer at 40° C. for 30 min, with continuous stirring until a clear liquid was observed. The following NADES were used for ethanol substitution tests:
BcC: coconut betaine, citric acid (molar ratio: 4:1).
CGIH: citric acid, glycerol, and water (molar ratio: 0,5:2,7:1)
BcS: coconut betaine, and sorbitol (molar ratio 1:4)
DgGI (1:3,5)
CDg (1:1,4)
DgGIC (1:1.8:2.1)

For the ethanol replacement test, NADES were used in the formulations containing different concentrations of the essential oil and water.

The procedure used involved reducing the percentage of ethanol in the different formulations to 10% and replacing the removed ethanol amount by an equivalent amount of the selected NADES. The mixtures were stirred thoroughly and then subjected to an ultrasonic bath at room temperature for 15 minutes.

The essential oil tested was:
Fragrance A: Midnight Lavender

After verifying that the NADES in the different formulations remained transparent and in a single phase, the NADES was used to decrease the percentage of ethanol and increase that of the NADES until equilibrium was reached.

The results of the mixtures are detailed in Table 3, in which the perfume formula is tested with various NADES.

Table 3 also shows the same similar replacement protocol, which is made using the NADES already previously mentioned, but rather than being used in the Eau de Parfume, the table shows the results in a Body Splash formulation that contains a higher percentage of water and a lower percentage of essential oils.

TABLE 3
Perfume formula, decrease in ethanol-increase in NADES.

| | Eau Parfum | | | | | Body Splash | | | | |
|---|---|---|---|---|---|---|---|---|---|---|
| | Ap | Bp | Cp | Dp | Ep | As | Bs | Cs | Ds | Es |
| Fragrance (%) | 15 | 15 | 15 | 15 | 15 | 3 | 3 | 3 | 3 | 3 |
| DPG (%) | 3 | 3 | 3 | 3 | 3 | 3 | 3 | 3 | 3 | 3 |
| Ethanol (%) | 74 | 7.4 | — | — | — | 73 | 7.3 | — | — | — |
| Aqua (%) | 7 | 7 | 7 | 7 | 7 | 20 | 20 | 20 | 20 | 20 |
| BcC | — | 66.6 | — | — | — | — | 66.7 | — | — | — |
| DgL | — | — | 74 | | | — | — | 73 | — | — |
| CDg | — | — | — | 74 | — | — | — | — | 73 | — |
| DgGlC | — | — | — | — | 74 | — | — | — | — | 73 |
| Preservative (%) | 1 | 1 | 1 | 1 | 1 | 1 | 1 | 1 | 1 | 1 |

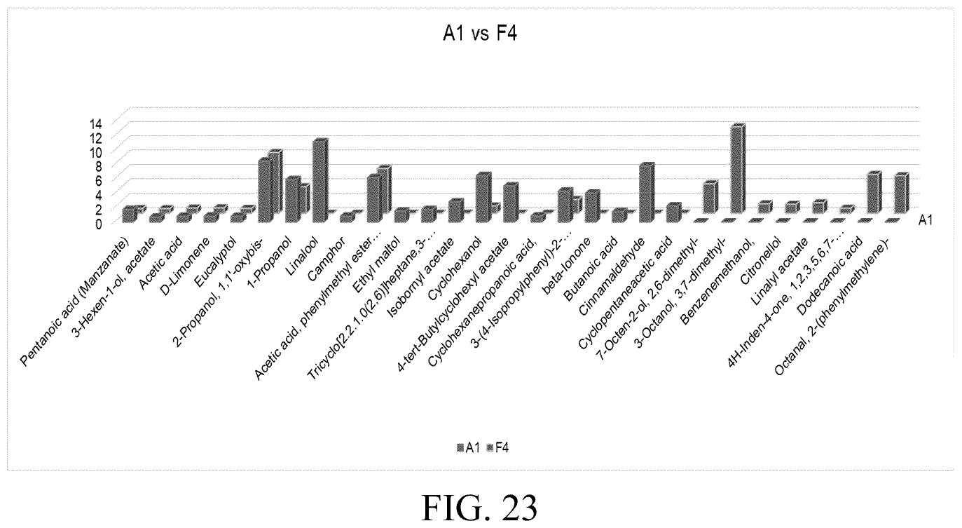

FIG. 23 shows a GC analysis of the Eau de parfum formulation: with A1 (green)) with BioE-HF-47, and F4 (orange) with 10% Ethanol.

The formulations also contained a pH regulator, which stabilizes the pH of cosmetics.

Triethanolamine (TEA) is a basic tertiary amine. It is often used as a pH adjuster or to thicken cosmetic formulas. Its manufacture results from the reaction of ethylene oxide (very toxic) with ammonia (also toxic). Ethylene oxide is implicated in possible damage to the liver and kidneys, and it may lead to respiratory irritation, lung injury, headaches, nausea, vomiting, diarrhea, shortness of breath, and cyanosis as well as increasing the probability of certain types of cancer. Ammonia may cause burning of the eyes, nose, throat, and respiratory tract and may result in blindness, lung damage, or even death.

In the presence of nitrite, TEA forms nitrosamines known to be carcinogenic. Nevertheless, people imagine that the risk remains very low. However, the results of many studies belie its safety as studies have shown that TEA is toxic to the skin, the immune system, and the respiratory tract in humans. One or more animal studies have shown effects on the sense organs even when administered at very low doses, especially when used around the mouth, eyes, and lips. In vitro cell tests showed that exposure to TEA in mammalian cells showed positive mutation results. Accordingly, its replacement by non-toxic NADES compositions would be highly innovative, generating a positive impact on the cosmetic industry.

Accordingly, the above experiments demonstrate that the general objectives of the present invention have been satisfied. That is, one was able to see enhanced solubility of essential oils within natural eutectic solvents (NADES) across various concentration levels. The mixture of essential oil/ES were solubilized in water at different concentration levels. Moreover, the present invention was able to assess the stability of the studied mixtures, and those experiments that show these results are enumerated below.

### Laboratory Test

NADES products were prepared by stirring in a container such as a flask with a magnetic stirrer at 40° C. for 30 min, with continuous stirring until the formation of a transparent liquid was observed. The following basic NADES were employed as pH regulators:
ArgCarH: L-arginine, L-carnitine, and water (molar ratio 1:0.6:83.2)
ArgMdH: L-arginine, maltodextrin, and water (molar ratio: 1:0.4:75.4)
ArgUH: L-arginine, urea, and water (molar ratio: 1:11.6:48.4)
LysXH (molar relation: 1:0.3:16)
LysXArgCarH (molar relation: 3.3:1:0.8:0.5:129.2)
   In this sense, basic NADES were tested to modulate pH in a base cream and a base gel.

In the case of the base cream, the pH was decreased with LGH (lactic acid, dextrose, and water (molar ratio of 5:1:9)) and a solution of citric acid at 30%. Then, the basic NADES were incorporated in the formulation at 0.5, 1, and 2% concentration levels. The results can be seen in Table 4.

pH variations with the addition of pH modifiers

| | Bioeutectics | pH | | | |
|---|---|---|---|---|---|
| Base cream | product | Initial | 0.5% | 1% | 2% |
| Base cream + BioE-F-01 | ArgCarH | 3.20 | 3.67 | 4.04 | 5.92 |
| Base cream + citric | | 3.30 | 3.80 | 3.96 | 5.90 |
| acid (30%) | | | | | |
| Base cream + BioE-F-01 | ArgMdH | 3.15 | 3.50 | 3.85 | 4.13 |
| Base cream + citric | | 3.25 | 3.97 | 4.79 | 5.95 |
| acid (30%) | | | | | |
| Base cream + BioE-F-01 | ArgUH | 3.20 | 3.74 | 4.13 | 6.55 |
| Base cream + citric | | 3.20 | 3.82 | 4.59 | 6.39 |
| acid (30%) | | | | | |
| Base cream + BioE-F-01 | LysXH | 3.21 | 3.75 | 9.31 | 9.89 |
| Base cream + citric | | 2.50 | 3.02 | 4.84 | 7.37 |
| acid (30%) | | | | | |
| Base cream + BioE-F-01 | LysXArgCarH | 3.03 | 7.89 | 9.50 | 10.10 |
| Base cream + citric | | 2.56 | 3.87 | 5.19 | 9.01 |
| acid (30%) | | | | | |
| Base cream + BioE-F-01 | TEA | 3.80 | 7.52 | 8.05 | 8.38 |
| Base cream + citric | | 3.50 | 6.95 | 7.64 | 8.29 |
| acid (30%) | | | | | |

It is important to note that the basic NADES showed an ability to affect the pH when the NADES is used at the 1-2% level without changing the appearance of the formulation. Creating a gel formulation with Carbopol typically involves a combination of ingredients to achieve the desired texture, viscosity, and stability. Here's a basic recipe for a Carbopol gel formulation: Distilled water, Carbopol polymer (e.g., Carbopol 940), Triethanolamine (TEA) or another suitable alkaline substance for pH adjustment, and preservative (e.g., parabens or phenoxyethanol).

In the case of base gels, the basic NADES were incorporated as pH regulators as well as gelling agents according to base formulations.

Correct formation of the gel was observed with a consistency typical of the formulation. The basic NADES give the formula other properties such as emollient and even antimicrobial power. The replacement of TEA in these cosmetic formulations was achieved successfully.

Thus, from the above experimental results, it should be apparent that NADES compositions may successfully be used in personal care products to afford personal care formulations that are less toxic while maintaining many of the desirable properties that are attained using traditional (and more toxic) formulations. By using NADES compositions in personal care products one is able to attain enhanced or good solubility, good stability, and also be able to adjust pH levels to be in the desirable ranges. The formulations with various commercially available essential oils demonstrate that there are NADES that can be used that show good solubility characteristics, NADES compositions that may be used that work at appropriate concentration levels, NADES compositions that can be used to appropriately adjust pH characteristics, and NADES compositions that give good emollient and anti-microbial properties. However, perhaps most importantly, the NADES compositions that are tested with personal care products as disclosed herein, demonstrate that the NADES compositions may be suitable for replacing their more dangerous typically used counterparts. These counterparts, in many instances, are known to have toxic characteristics.

In an embodiment, the present invention relates to a personal care product formulation that comprises an active ingredient and a NADES composition. In a variation, the NADES composition comprises one or more of a) L-arginine, L-carnitine, and water, b) L-arginine, maltodextrin, and water, c) L-arginine, urea, and water, d) L-arginine, glycerol, and water, e) lactic acid, dextrose, and water, f) citric acid, glycerol, and water, betaine and lactic acid, g) glucose, glycerol, and water, h) L-arginine, lactic acid, and water, i) L-arginine, citric acid, and water, j) betaine, urea, and water, or k) coconut betaine and citric acid.

In a variation, the NADES composition provides solubility or stability of the active ingredient or adjusts a pH of the formulation comprising the active ingredient and the NADES composition. In a variation, the active ingredient provides one or more of the following desired characteristics: desired fragrance, desired viscosity, desired moisturization, desired cleansing, desired emulsification, desired stability, desired solubility, desired non-toxicity, desired smoothing ability, desired softening ability, desired emollient properties, desired anti-aging ability, desired wrinkle prevention ability, desired lubrication ability, desired anti-oxidant ability, desired lather ability, desired property allowing the active ingredient to be removed or retained on the applied site, desired protectant ability like UV blocking agents, desired graininess, desired anti-microbial ability, desired tint or color, desired opaqueness or transparency, desired thickening or lengthening ability, desired luster ability, desired antiperspirant or deodorant ability, desired sweat gland blocking ability, desired disease treating ability, desired grittiness or abrasiveness, desired flavor, desired evaporation or degradation characteristics, or desired misting or nebulization ability.

In a variation, the desired anti-microbial ability comprises antifungal, antibacterial, or antiviral activity. In a variation, the NADES composition comprises coconut betaine and citric acid. In a variation, the molar ratio of the coconut betaine and citric acid is about 1 to 2.

In a variation, the active ingredient is an essential oil or a fragrance. In a variation, the formulation is used in one or more colognes, perfumes, mouthwashes, deodorants, toothpastes, hair gels, shampoos, conditioners, body washes, hair removal creams, lotions, hand creams, eye creams, shaving creams, cleansers, eye drops, soaps, sunscreens, exfoliators, sera, or moisturizers. In a variation, the fragrance is Midnight Lavender, Free Spirit, Arabian Jasmine, Fir bark, Jack Pine or Black Spruce.

In a variation, the pH is adjusted to a pH greater than 4, or 5, or 6, or 7, or 8. In a variation, the solubility of the active ingredient is at least about 10% w/w, or 20% w/w, or 30% w/w, or 40% w/w, or 50% w/w.

In an embodiment, the present invention relates to a method of reducing toxicity of a personal care product formulation that comprises triethanolamine and an active ingredient, said method comprising replacing the triethanolamine with a NADES composition. In a variation of the method, the NADES composition comprises one or more of a) L-arginine, L-carnitine, and water, b) L-arginine, maltodextrin, and water, c) L-arginine, urea, and water, d) L-arginine, glycerol, and water, e) lactic acid, dextrose, and water, f) citric acid, glycerol, and water, betaine and lactic acid, g) glucose, glycerol, and water, h) L-arginine, lactic acid, and water, i) L-arginine, citric acid, and water, j) betaine, urea, and water, or k) coconut betaine and citric acid.

In a variation, the active ingredient provides one or more of the following desired characteristics: desired fragrance, desired viscosity, desired moisturization, desired cleansing, desired emulsification, desired stability, desired solubility, desired non-toxicity, desired smoothing ability, desired softening ability, desired anti-aging ability, desired wrinkle prevention ability, desired lubrication ability, desired anti-oxidant ability, desired lather ability, desired property allowing the active ingredient to be removed or retained on the applied site, desired protectant ability like UV blocking agents, desired graininess, desired anti-microbial ability, desired tint or color, desired opaqueness or transparency, desired thickening or lengthening ability, desired luster ability, desired antiperspirant or deodorant ability, desired sweat gland blocking ability, desired disease treating ability, desired grittiness or abrasiveness, desired flavor, desired evaporation or degradation characteristics, or desired misting or nebulization ability.

In a variation, the NADES comprises one or more of a) L-arginine, L-carnitine, and water, b) L-arginine, maltodextrin, and water, c) L-arginine, urea, and water, or d) L-arginine, glycerol, and water.

In a variation, the personal care product formulation is used one or more of colognes, perfumes, mouthwashes, deodorants, toothpastes, hair gels, shampoos, conditioners, body washes, hair removal creams, lotions, hand creams, eye creams, shaving creams, cleansers, eye drops, soaps, sunscreens, exfoliators, sera, or moisturizers.

In a variation, the active ingredient is an essential oil or a fragrance. In a variation, the desired anti-microbial ability comprises antifungal, antibacterial, or antiviral activity.

In an embodiment, the present invention relates to a NADES composition that is selected from the group consisting of: a) L-arginine, L-carnitine, and water, b) L-arginine, maltodextrin, and water, c) L-arginine, urea, and water, d) L-arginine, glycerol, and water, e) lactic acid, dextrose, and water, f) citric acid, glycerol, and water, betaine and lactic acid, g) glucose, glycerol, and water, h) L-arginine, lactic acid, and water, i) L-arginine, citric acid, and water, j) betaine, urea, and water, and k) coconut betaine and citric acid. In a variation, the NADES has no other ingredients in it.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

We claim:

1. A personal care product formulation that comprises an active ingredient and a NADES composition.

2. The personal care product of claim 1, wherein the NADES composition comprises one or more of a) L-arginine, L-carnitine, and water, b) L-arginine, maltodextrin, and water, c) L-arginine, urea, and water, d) L-arginine, glycerol, and water, e) lactic acid, dextrose, and water, f) citric acid, glycerol, and water, g) betaine and lactic acid, h) glucose, glycerol, and water, i) L-arginine, lactic acid, and water, j) L-arginine, citric acid, and water, k) betaine, urea, and water, l) coconut betaine and citric acid, m) decyl glucoside and lactic acid, n) citric acid and dodecyl glucoside, or o) decyl glucoside, glycerol, and citric acid.

3. The personal care product formulation of claim 1, wherein the NADES composition provides solubility or stability of the active ingredient, or adjusts a pH of the formulation comprising the active ingredient and the NADES composition.

4. The personal care product formulation of claim 1, wherein the active ingredient provides one or more of the following desired characteristics: desired fragrance, desired viscosity, desired moisturization, desired cleansing, desired emulsification, desired stability, desired solubility, desired non-toxicity, desired smoothing ability, desired softening ability, desired anti-aging ability, desired wrinkle prevention ability, desired lubrication ability, desired anti-oxidant ability, desired lather ability, desired property allowing the active ingredient to be removed or retained on the applied site, desired protectant ability like UV blocking agents, desired graininess, desired anti-microbial ability, desired tint or color, desired opaqueness or transparency, desired thickening or lengthening ability, desired luster ability, desired antiperspirant or deodorant ability, desired sweat gland blocking ability, desired disease treating ability, desired grittiness or abrasiveness, desired flavor, desired evaporation or degradation characteristics, or desired misting or nebulization ability.

5. The personal care product formulation of claim 4, wherein the desired anti-microbial ability comprises antifungal, antibacterial, or antiviral activity.

6. The personal care product formulation of claim 2, wherein the NADES composition comprises coconut betaine and citric acid.

7. The personal care product formulation of claim 6, wherein a molar ratio of the coconut betaine and citric acid is about 1 to 2.

8. The personal care product formulation of claim 1, wherein the active ingredient is an essential oil or a fragrance.

9. The personal care product formulation of claim 1, wherein the formulation is used in one or more of colognes, perfumes, mouthwashes, deodorants, toothpastes, hair gels, shampoos, conditioners, body washes, hair removal creams, lotions, hand creams, eye creams, shaving creams, cleansers, eye drops, soaps, sunscreens, exfoliators, sera, or moisturizers.

10. The personal care product formulation of claim 8, wherein the fragrance is Midnight Lavender, Free Spirit, Fir bark, Jack Pine, Black Spruce or Arabian Jasmine.

11. The personal care product formulation of claim 3, wherein the pH is adjusted to a pH greater than 4.

12. The personal care product formulation of claim 3, wherein the solubility of the active ingredient is at least about 10% w/w.

13. A method of reducing toxicity of a personal care product formulation that comprises triethanolamine and an active ingredient, said method comprising replacing the triethanolamine with a NADES composition.

14. The method of claim 13, wherein the NADES composition comprises one or more of a) L-arginine, L-carnitine, and water, b) L-arginine, maltodextrin, and water, c) L-arginine, urea, and water, d) L-arginine, glycerol, and water, e) lactic acid, dextrose, and water, f) citric acid, glycerol, and water, g) betaine and lactic acid, h) glucose, glycerol, and water, i) L-arginine, lactic acid, and water, j) L-arginine, citric acid, and water, k) betaine, urea, and water, l) coconut betaine and citric acid, m) decyl glucoside and lactic acid, n) citric acid and dodecyl glucoside, or o) decyl glucoside, glycerol, and citric acid.

15. The method of claim 13, wherein the active ingredient provides one or more of the following desired characteristics: desired fragrance, desired viscosity, desired moisturization, desired cleansing, desired emulsification, desired stability, desired solubility, desired non-toxicity, desired smoothing ability, desired softening ability, desired anti-aging ability, desired wrinkle prevention ability, desired lubrication ability, desired anti-oxidant ability, desired lather ability, desired property allowing the active ingredient to be removed or retained on the applied site, desired protectant ability like UV blocking agents, desired graininess, desired anti-microbial ability, desired tint or color, desired opaqueness or transparency, desired thickening or lengthening ability, desired luster ability, desired antiperspirant or deodorant ability, desired sweat gland blocking ability, desired disease treating ability, desired grittiness or abrasiveness, desired flavor, desired evaporation or degradation characteristics, or desired misting or nebulization ability.

16. The method of claim 13, wherein the NADES comprises one or more of a) L-arginine, L-carnitine, and water, b) L-arginine, maltodextrin, and water, c) L-arginine, urea, and water, or d) L-arginine, glycerol, and water.

17. The method of claim 13, wherein the personal care product formulation is used one or more of colognes, perfumes, mouthwashes, deodorants, toothpastes, hair gels, shampoos, conditioners, body washes, hair removal creams, lotions, hand creams, eye creams, shaving creams, cleansers, eye drops, soaps, sunscreens, exfoliators, sera, or moisturizers.

18. The method of claim 13, wherein the active ingredient is an essential oil or a fragrance.

19. The method of claim 15, wherein the desired anti-microbial ability comprises antifungal, antibacterial, or antiviral activity.

20. A NADES composition that is selected from the group consisting of: a) L-arginine, L-carnitine, and water, b) L-arginine, maltodextrin, and water, c) L-arginine, urea, and water, d) L-arginine, glycerol, and water, e) lactic acid, dextrose, and water, f) citric acid, glycerol, and water, g) betaine and lactic acid, h) glucose, glycerol, and water, i) L-arginine, lactic acid, and water, j) L-arginine, citric acid, and water, k) betaine, urea, and water, l) coconut betaine and citric acid, m) decyl glucoside and lactic acid, n) citric acid and dodecyl glucoside, and o) decyl glucoside, glycerol, and citric acid.
